(19) United States
(12) Patent Application Publication
(10) Pub. No.: US 2002/0045172 A1
(43) Pub. Date: Apr. 18, 2002

(54) Segmented support structure and method and fixture for making the same

(76) Inventors: Albert J. Sturm JR., Stillwater, MN (US); Fred W. Hanson, White Bear Lake, MN (US)

Correspondence Address: Steven M. Koehler, WESTMAN CHAMPLIN & KELLY, International Centre, Suite 1600, 900 South Second Avenue, Minneapolis, MN, 55402-3319, US

(21) Appl. No.: 09/829,269

(22) Filed: Apr. 9, 2001

Related U.S. Application Data
(60) Provisional application No. 60/215,560, filed on Jun. 30, 2000.

(51) Int. Cl. C12Q 1/68

(52) U.S. Cl. 435/6

(57) ABSTRACT

A gantry segmented support structure is provided with overlapping wall plates that can include a damping material disposed therebetween. The structure forms a tube assembly, which when configured with the damping material is adapted to dampen forces resulting from performing operations on a workpiece. In particular, the damping prevents unwanted oscillatory displacement of the segmented support structure. The overlapping wall plates allow a housing created thereby to be attached to a rigid plate without inducing strain in the rigid plate that can deform the rigid plate. This construction eliminates manufacturing of bulky weldments, which thereby reduces costs. A method of assembly and a fixture are also provided.

CROSS-REFERENCE TO RELATED PATENT APPLICATION

This application claims the benefit of U.S. provisional patent application Ser. No. 60/215,560, filed on Jun. 30, 2000, the content of which is hereby incorporated by reference in its entirety.

### BACKGROUND OF THE INVENTION

The present invention relates to support structures used in gantry systems. More particularly, the present invention relates to gantry support structures having improved damping characteristics and/or are easier to manufacture.

Gantry systems are well known and commonly used in tooling, milling or positioning systems. Generally, the gantry system includes two spaced apart rails. A bridge extends between the rails and travels thereon. A mast is coupled to the bridge. The mast supports milling tools, lifting devices, sensors or the like. Typically, the mast is movable along the bridge and telescopes in order to provide three degrees of freedom, while an end effector mounted at the end of the mast can provide additional movements.

In many applications, such as lifting or milling operations, it is necessary that the gantry system be able to position a device such as a tool accurately. However, unwanted oscillatory displacement can occur in certain operating conditions. In particular, components of the gantry system may resonate during operation, for example, during starting and stopping movements by the bridge or mast.

In addition to the foregoing, current gantry bridges are expensive to manufacture. Commonly, the bridge is welded from a number of components. The bulky weldment then must be machined to form a mounting surface suitable for guiding elements of the mast. In many cases, the bridge must be sent to a remote location for machining. Thus, besides the machining costs, significant transportation costs are also incurred.

An improved gantry system that addresses one or more of the above-described problems is therefore needed.

### SUMMARY OF THE INVENTION

A first aspect of the present invention includes a gantry segmented support structure having overlapping wall panels or plates with a damping material disposed therebetween. The structure forms a tube assembly and is adapted to dampen forces resulting from performing operations on a workpiece. In particular, the damping material reduces unwanted oscillatory displacement of the segmented support structure. The gantry segmented support structure can be embodied in a bridge or mast. A method of making the same and a fixture for making the same embody other aspects of the present invention.

A further aspect of the present invention includes a gantry system having spaced apart guide rails, a mast and a bridge extending between and supported on the guide rails for movement therealong. The bridge includes a plate with a mounting surface for supporting the mast and a segmented assembly joined to the plate to form an enclosed perimeter with the plate without deforming the mounting surface when the segmented assembly is joined to the plate. The foregoing bridge structure significantly reducing manufacturing costs because expensive machining of the bridge structure after construction is not necessary.

### BRIEF DESCRIPTION OF THE DRAWINGS

FIG. 1 is a side view of a gantry system.

FIG. 2 is a top view of a portion of the gantry system.

FIG. 3 is a front view of a portion of the gantry system.

FIG. 4 is a cross-sectional view taken along the line 4-4 of FIG. 2.

FIG. 5 is a cross section of a segmented support structure.

FIG. 6 is a rear elevational view of a mast of the gantry system.

FIG. 7 is a front elevation view of an alternative mast of the gantry system.

FIG. 8 is a partial view of wall plates of the gantry system.

FIG. 9 is an exploded perspective view of a fixture for forming the segmented support structure.

FIG. 10 is a front elevational view of the fixture.

FIG. 11 is a top plan view of an end truck that supports the bridge and is carried by a guide rail.

FIG. 12 is a sectional view taken along lines 12-12 of FIG. 11 illustrating a damping assembly for supporting the end truck on the guide rail.

### DETAILED DESCRIPTION OF THE ILLUSTRATIVE EMBODIMENTS

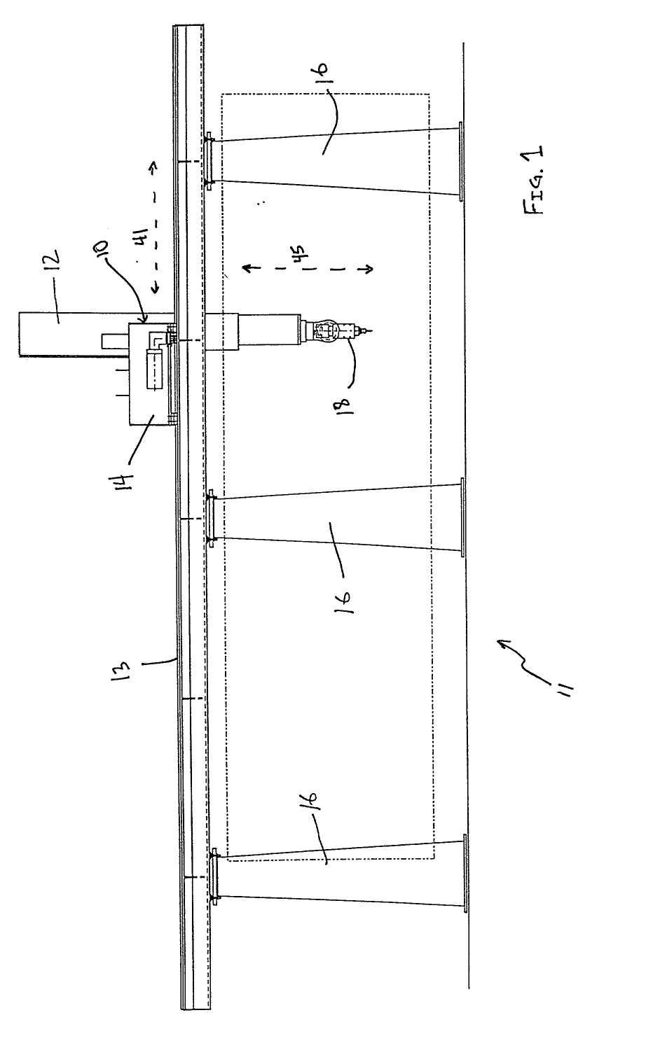

Referring to FIGS. 1-3, a gantry system is illustrated at 11. Gantry system 11 includes a pair of spaced apart rails 13. In the exemplary embodiment, rails 13 are elevated, being supported by supports 16. Bridge 10 spans between rails 13. Two trucks 14 are coupled to bridge 10 and coupled to rails 13 in order to provide horizontal movement of bridge 10 in a direction parallel to axis 41 as is well known in the art. Bridge 10 supports a mast 12, which is adapted to hold an end effector 18 such as a tool, lifting device, sensor or the like. Bridge 10 can be adapted to allow movement of mast 12 along bridge 10 parallel to the direction of axis 43. In the embodiment illustrated, mast 12 comprises a telescoping assembly in order to allow end effector 18 to move in a direction parallel to axis 45. Typically, axes 41, 43 and 45 are orthogonal to each other, thereby allowing three-dimensional movement of the end effector 18.

At this point, it should be noted that the general design of the trucks 14 and rails 13, which allows movement of the trucks on the corresponding rails 13, does not form part of the present invention and can take any of many known forms. However, damping can be provided in the trucks as described below as another aspect of the present invention. Displacement of the mast 12 on the bridge 10 can also be performed using conventional displacement mechanisms and also does not form part of the present invention. However, as discussed below, the design of bridge 10 allows accurate positioning of mast 12 on bridge 10 and can reduce manufacturing or construction costs.

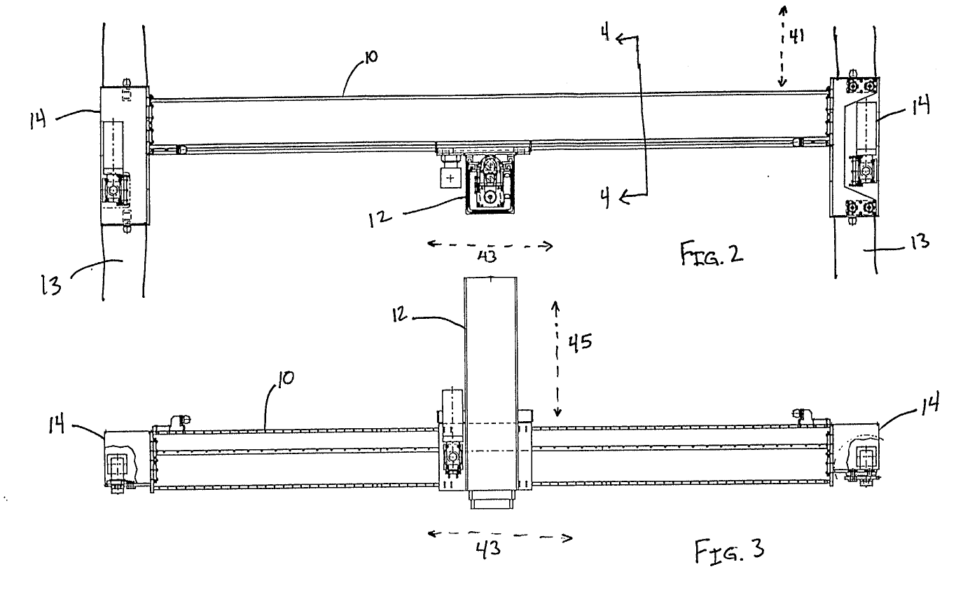

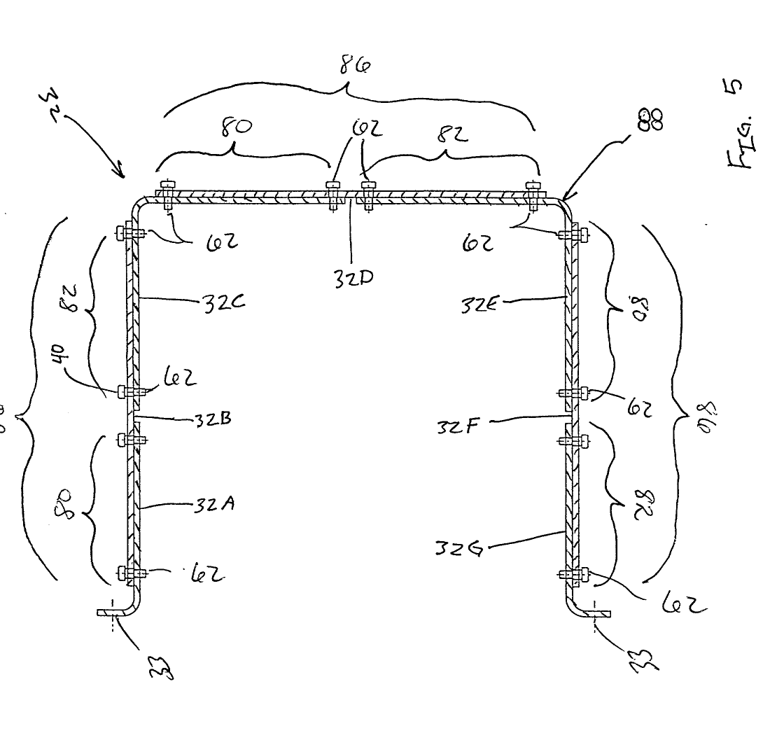

FIG. 4 illustrates a cross-sectional view taken along the line 4-4 of FIG. 2. Bridge 10 is mounted to each truck 14 with mounting flanges 22, 24, 26 and 28. The mounting flanges can be blocks of material with mounting bores extending therethrough. A plurality of fasteners 42 extend through a first set of apertures of mounting flanges 22, 24, 26 and 28 and secure mounting flanges 22, 24, 26 and 28, and thus, bridge 10 to each truck 14. A second plurality of fasteners 43 extend through a second set of apertures (orthogonal to the first set of apertures) and secure edges of the bridge 10 to the mounting flanges 22, 24, 26 and 28.

Generally, bridge 10 includes a segmented assembly 32 (described below) attached to a rigid plate 30 with a plurality of fasteners 38 extending along the length of bridge 10. Segmented assembly 32 forms a portion of a housing. Segmented assembly 32 is U-shaped and adapted to fasten to a major surface 30A of rigid plate 30. As illustrated in this embodiment, segmented assembly 32 includes a plurality of flanges 33 to fasten to surface 30A of rigid plate 30. Linear bearings 36 are provided to support mast 12 and allow movement of mast 12 along bridge 10. Linear bearings 36 can be coupled to a major surface 30B of rigid plate 30 as illustrated. In a further embodiment, fasteners 38 can pass through apertures in rigid plate 30 in order to secure segmented assembly 32 and linear bearings 36 together on opposite sides of rigid plate 30. Thus, the amount of machining of rigid plate 30 is reduced.

It is important to note that this construction significantly reduces costs of gantry bridges. Rigid plate 30 can be pre-machined to form major surface 30B suitable for mounting of the linear bearings 36. Segmented assembly 32 is formed separately with mounting portions 33 for securing to rigid plate 30 configured to closely correspond to the surface of rigid plate 30 to which it is to be secured. This construction significantly reduces the manufacturing cost because in prior art techniques of bridge construction after the bridge is formed, the bridge must be then machined on one or more surfaces for the mast guide assemblies. However in the present invention, by configuring the portions 33 of assembly 32 to correspond to rigid plate 30, prior to assembly, this construction allows the assembly 32 to be then secured to rigid plate 30 without inducing any twist in rigid plate 30, which would cause corresponding distortion in the major surface 30B. In this manner, only a simple plate 30 needs to be machined, and in fact, can be purchased as a stock item. Whereas, in the prior art techniques, a large, bulky weldment would need to be machined, often at a remote site, thereby incurring significant transportation costs.

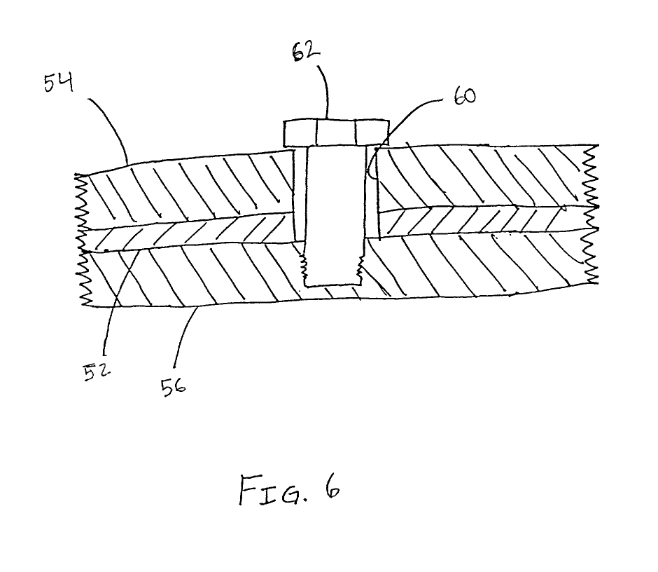

In the embodiment illustrated, assembly 32 is formed from overlapping segments. FIG. 5 shows a cross section of segmented assembly 32 of bridge 10. Overlapping segments or plates 32A-32G are adapted to form a perimeter wall or a portion of the overall housing. Segments 32A-32G are fastened together along a length of the segmented assembly 32 or bridge 10. Segments 32A and 32G include edges or portions 33, herein flanges, that are adapted to correspond to surface 30A of rigid plate 30 (illustrated in FIG. 4). In particular, the overlap of successive segments allows the edges 33 to correspond to surface 30A. When adjusted, the segments can be secured together with a suitable fastening device such as welding, brazing, mechanical fasteners (nuts and bolts), etc. herein an adhesive that bonds the segments together. For the reasons discussed above, the adhesive can comprise the damping material to damp the bridge in operation; however, this should be understood as one form of adhesive that can have beneficial characteristics.

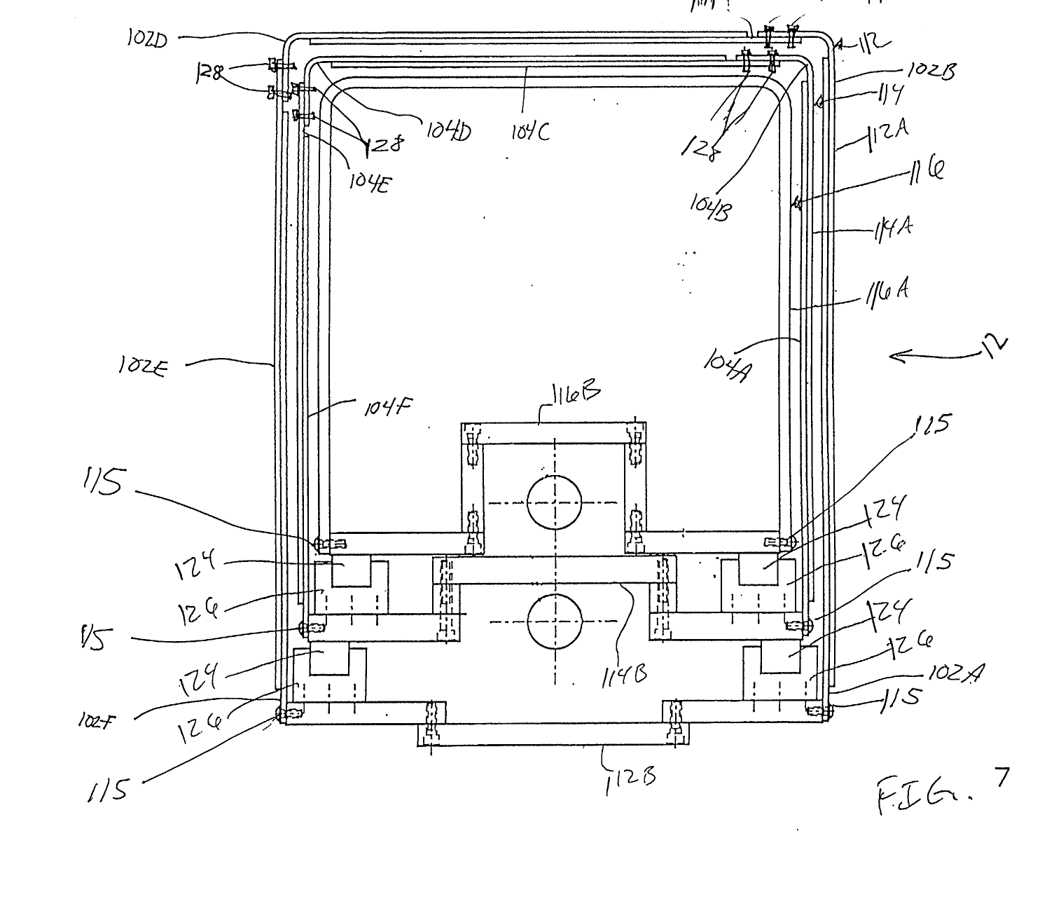

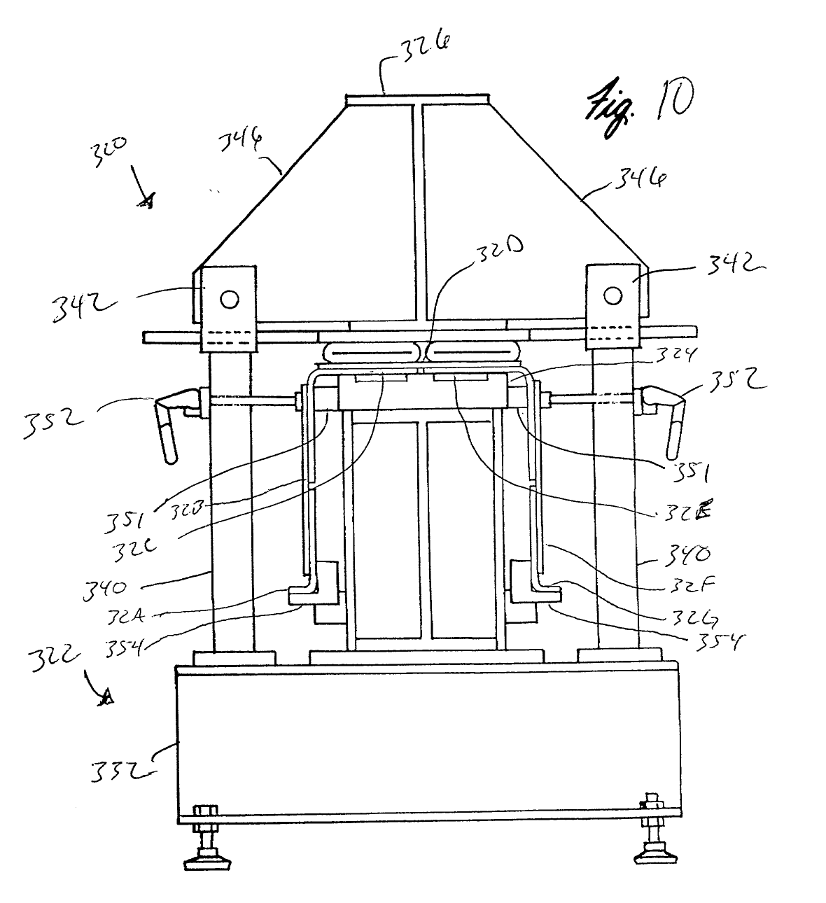

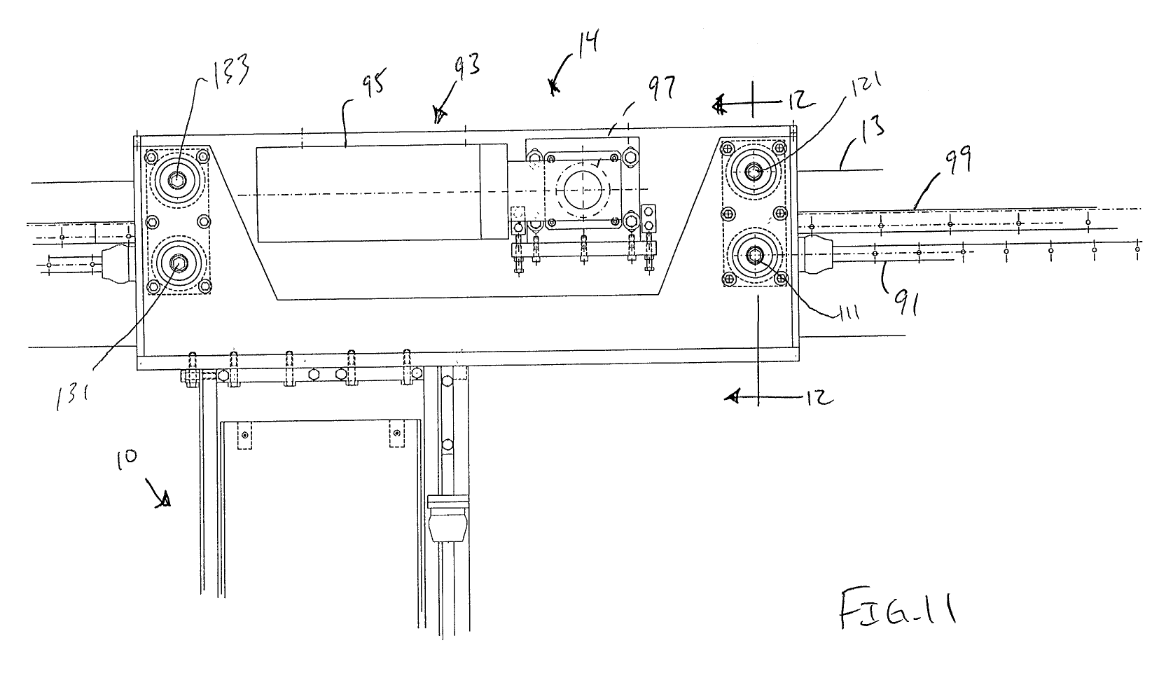

Fastening of the segments with damping material is more clearly illustrated in FIG. 6. Generally, in order to provide damping of unwanted oscillatory displacement of the bridge 10 and mast 12, discussed below, a perimeter wall of the bridge 10 or mast 12 is formed of overlapping wall plates with damping material disposed therebetween. FIG. 6 is an exemplary view of this construction. Damping material 52 has adhesive on two sides in order to fasten wall plates or segments 54 and 56 together. Wall plates 54 and 56 overlap each other for added strength. Damping material 52 is disposed between wall plates 54 and 56 and can be a viscoelastic damping polymer manufactured by 3M Corporation of St. Paul, Minn. or any similar material. In short, damping material 52 increases the stiffness of the bridge 10 or mast 12. Generally, the wall plates are clamped together with the adhesive damping material disposed therebetween. A fixture 340 illustrated in FIGS. 9 and 10 and discussed below is used to form the segmented assembly of bridge 10 or mast 12.

If desired, clearance hole 60 can be provided to receive overtravel stop 62. Overtravel stop 62 can be a rod or a bolt extending between the overlapping plates 54 and 56. In the embodiment illustrated, overtravel stop 62 is fastened to wall plate 56 to limit relative movement between wall plates 54 and 56. Overtravel stops 62 can be provided throughout the segmented construction of bridge 10 and/or mast 12.

Referring back to FIG. 5, this figure also illustrates fastening regions 80 and 82 defined by the adhesive bonding areas between successive plates. Fastening regions 80 and 82 are spaced apart proximate a middle of each side of segmented assembly 32 such as side 86. As appreciated by those skilled in the art, spaced apart fastening regions can be provided at other locations around the perimeter of segmented assembly 32. For instance, fastening regions can also be spaced apart proximate a corner 88 of the segmented assembly 32. Similar fastening regions are located around the perimeter of the structure formed by segments 32A-32G. The fastening regions 80 and 82 extend along the length of the bridge 10, thereby eliminating the need for additional fasteners such as bolts. As a result, the construction of the tubular section is simplified and manufacturing costs are reduced.

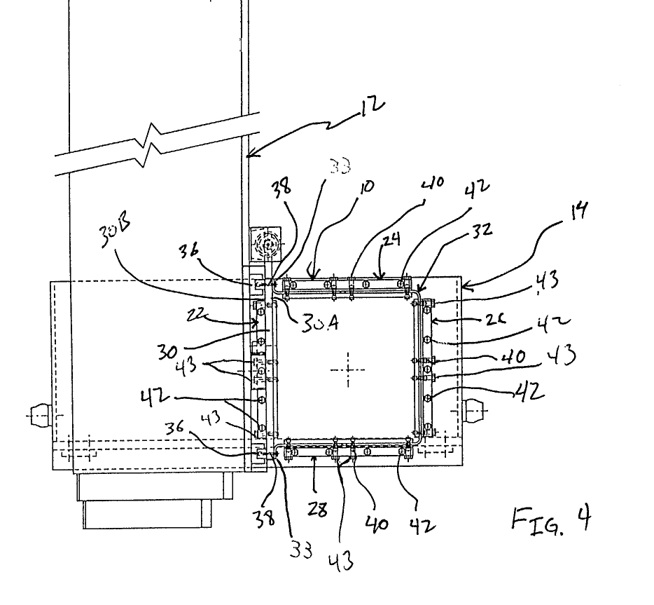

Bridge 10 with the construction described above is well suited for damping unwanted oscillatory wide displacement in many modes; however, bridge 10 is relatively stiff or ridged along its length. In other words, bridge 10 is relatively stiff in the direction indicated by double arrow 43 in FIG. 3. As another aspect of the present invention, further damping can be provided to control this mode of displacement.

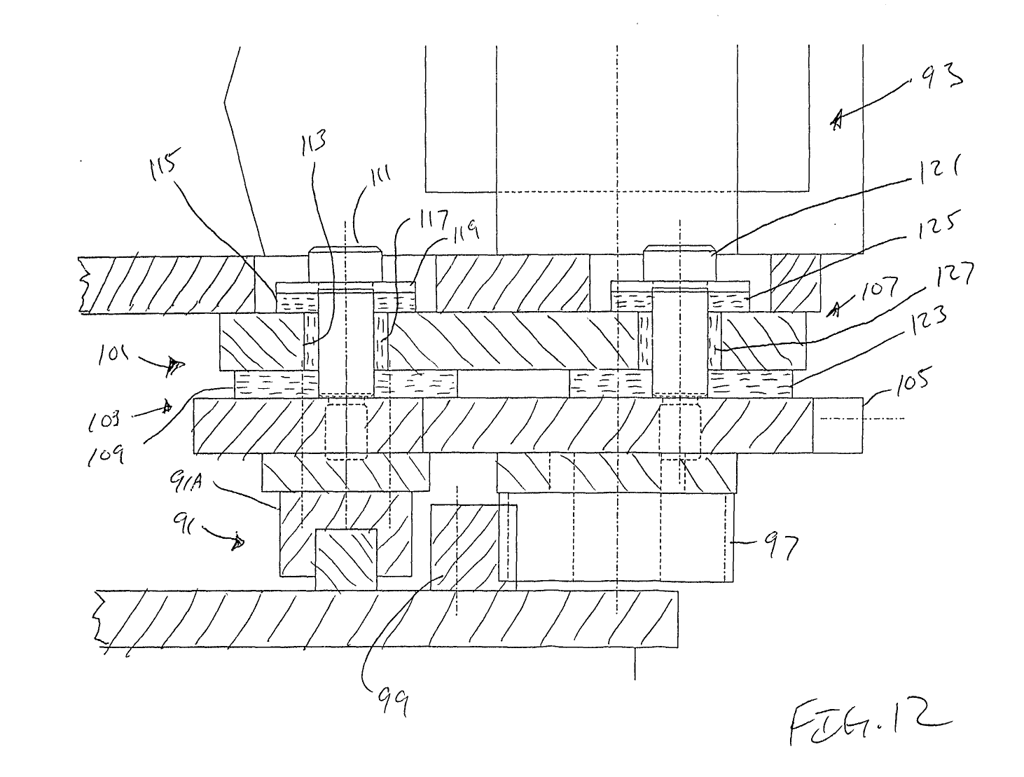

Referring now to FIG. 11, an enlarged plan of one of the trucks 14 is illustrated. As is common in the art, truck 14 is supported on and is guided along rail 13 by a guide assembly such as a linear bearing 91 that runs along the length of rail 13. Truck 14 commonly includes a drive assembly 93 having a motor 95 and a pinion gear 97. Pinion gear 97 engages a linear gear rack 99 that also extends along the length of guide rail 13. Selective operation of the drive assembly 93 causes rotation of the pinion gear 97 to move truck 14 as desired along rail 13. A similar drive assembly 93, linear bearing 91 and gear rack 99 are provided at the other end of mast 10. However, typically only one of the trucks 14 are restrained in a direction perpendicular to rail 13 such that the truck 14 is accurately moved along a selected path. Although the other truck is also guided on a linear bearing, the other truck is allowed to float in a direction perpendicular to rail 13 such that it is not necessary to ensure that the linear bearings on each of the guide rails are perfectly parallel to each other. In the embodiment illustrated and discussed below, damping is provided on truck 14 that is unrestrained or allowed to float; however, it should be understood that the damping assembly can be provided on the restrained truck, or on both trucks 14, if desired.

FIG. 12 is a sectional view illustrating a damping mounting assembly 101 provided on truck 14. Damping assembly 101 includes an elastomeric elements 103 that isolate portions of truck 14 so as to provide compliance and thus damping. In the embodiment illustrated, truck 14 includes a support plate 105 that supports a truck portion 91A of linear bearing 91. Support plate 105 is separated from a base assembly 107 of truck 14 by a damping element 109, herein a disc or washer that encircles a mounting bolt 111. In the embodiment illustrated, mounting bolt 111 extends through an aperture 113 in base assembly 107 to threadably engage a threaded aperture in support plate 105. As illustrated, two additional damping elements 115 and 117 can also be provided to improve isolation. Damping element 115 also comprises a washer that is disposed between base assembly 107 and a mounting washer 119 of mounting bolt 111. Damping element 117 is a cylindrical element that isolates a shaft of the mounting bolt 111 from the base assembly 107. Each of the damping elements 109, 115 and 117 can be made of any suitable material that provides compliance. One type of material includes fabric reinforced neoprene having fabric layers oriented as illustrated with dashed lines. Such elements are available, for example, from Fabreeka International, Inc. of Stoughton, Mass., U.S.A.

In the embodiment illustrated, a second mounting bolt 121 is similarly used with damping elements 123, 125 and 127. Referring back to FIG. 11, four mounting bolts 111, 121, 131 and 133 can be used with the construction described above to provide stability and damping for truck 14.

If desired, drive assembly 93 can also be provided with isolation mounts to minimize vibrations.

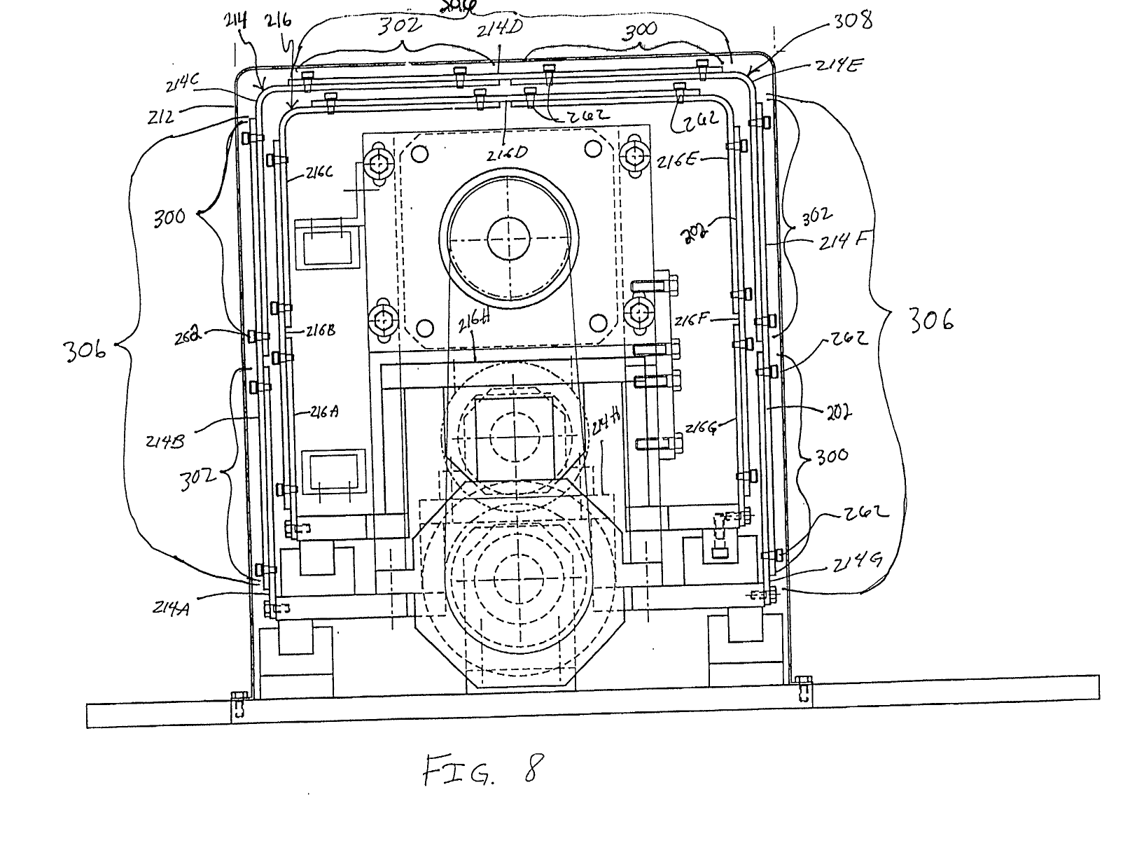

FIG. 7 illustrates an embodiment of a telescoping mast 12 as an aspect of the present invention. The mast 12 includes longitudinal segmented assembly tube 112 having first and second wall plates 102A and 102F defining first and second spaced apart longitudinal edges. A rigid plate 112B is joined to the spaced apart longitudinal edges with fasteners 115. A first L-shaped wall plate 102B is fastened to the first wall plate 102A with damping material 202 disposed therebetween as illustrated in FIG. 6. A third wall plate 102C is fastened with bolts 128 to the first L-shaped wall plate 102B and fastened to a second L-shaped wall plate 102D with damping material 202 disposed therebetween. A fourth wall plate 102E is fastened to the second L-shaped wall plate 102D with bolts 128 and is also fastened to the second wall plate 102F with damping material 202 disposed therebetween as discussed above. Segmented tube assembly 114 is similarly constructed with overlapping wall plates 104A-104F, fastened together to form a housing 114A and joined to rigid plate 114B with fasteners 115. In the embodiment illustrated, segmented tube assembly 116 comprises housing 116A and rigid plate 116B, but could be constructed in a manner similar to segmented tube assembly 114. In this embodiment, segmented tube assemblies 114 and 116 are movable relative to tube section 112, which is fixed with respect to axis 45. Bolts 128 and fasteners 115 are placed along the length of tube assemblies 112 and 114 to help hold the sections together. In effect bolts 128 form two large L shaped corner assemblies from plates 102B and 102C, and plates 102D and 102E, which could also be integrally formed together.

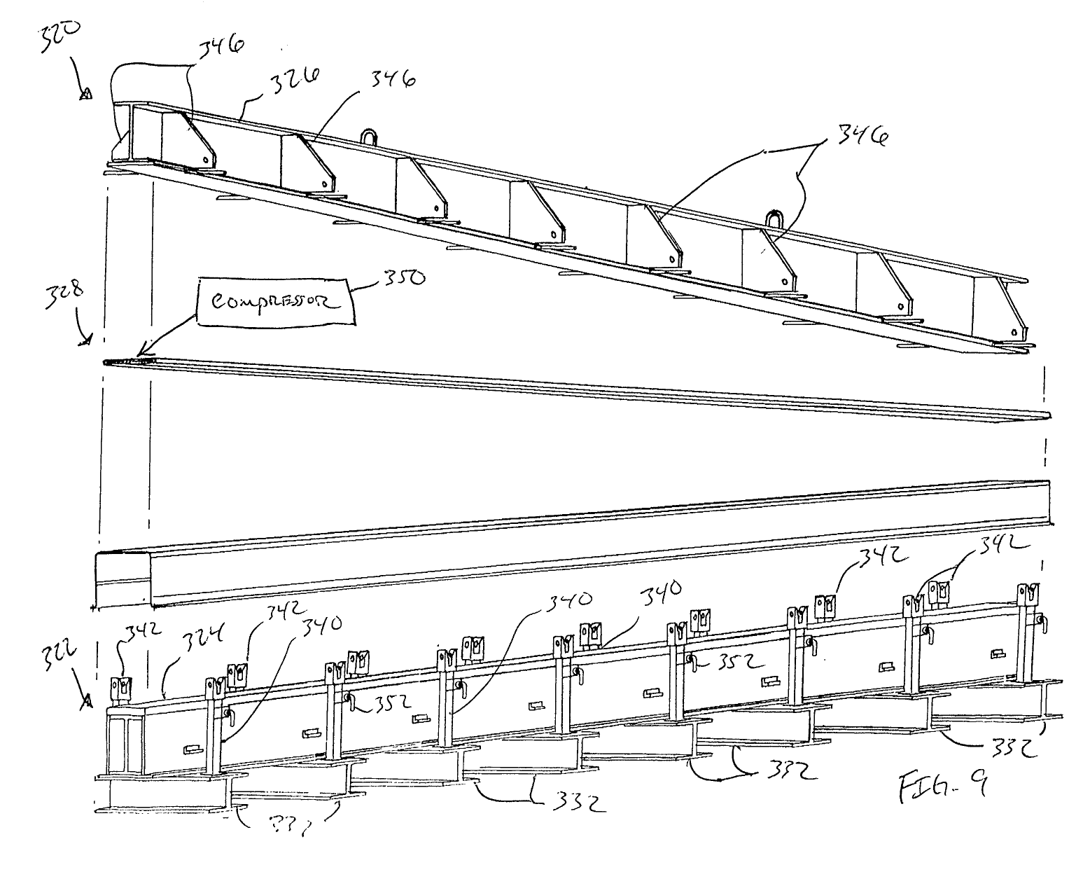

FIG. 8 is a front elevation view of a mast of an alternative embodiment of the present invention. Longitudinal section 212 surrounds segmented tube assemblies 214 and 216. Segmented tube assembly 214 surrounds segmented tube assembly 216. Segmented tube assembly 214 and segmented tube assembly 216 are movable relative to each other and longitudinal section 212 and are comprised of wall plates 214A-214G and 216A-216G, respectively. Wall plates 214A-214G and 216A-216G are fastened together in the manner illustrated in FIG. 6 with adhesive placed on both sides of a damping material as discussed above. Overtravel stops 262 are provided in order to limit relative movement between the adhesively joined wall plates.

Wall plates 214A and 214G define longitudinal edges of segmented tube assembly 214 and are joined to rigid plate 214H. Wall plate 214B joins wall plate 214A and L-shaped wall plate 214C with damping material disposed therebetween. Wall plate 214D joins L-shaped wall plates 214C and 214E together with damping material disposed therebetween. Wall plate 214F in turn joins together L-shaped wall plate 214E and wall plate 214G with damping material disposed therebetween. A similar structure is applied to tube assembly 216.

FIG. 8 also illustrates by way of example fastening regions 300 and 302. Fastening regions 300 and 302 are spaced apart proximate a middle of each side of segmented tube assembly 214 such as side 306. As appreciated by those skilled in the art, spaced apart fastening regions can be provided at other locations around the perimeter of the tube. For instance, fastening regions can also be spaced apart proximate a corner 308 of the segmented tube assembly 214. Similar fastening regions are located around the perimeter of the structure formed by wall plates 214A-214G. The fastening regions 300 and 302 extend along the length of the tube sections thereby eliminating the need for additional fasteners such as bolts in the previous embodiment. As a result, the construction of the tube section is simplified and manufacturing costs are reduced.

Bridge 10 or mast 12 can be fairly long in length. For instance, in one embodiment, bridge 10 can be fifteen feet long or longer, while each tubular section of mast 12 can be six feet long or longer. As described above, bridge 10 or mast 12 can be formed of plates adhesively joined together, for instance, with the viscoelastic damping polymer material disposed therebetween. However, a significant problem can be ensuring sufficient contact exists between the bonding material and each of the adjoining plates along the surface of the plates. FIGS. 9 and 10 illustrate a unique fixture 320 for forming the segmented assemblies. Generally, fixture 320 includes a base assembly 322 having an elongated support surface 324; an elongated reaction structure 326 held fixed relative to the support surface 324; and an elongated inflatable member 328 disposed between the support surface 324 and the reaction structure 326 along the support surface 324 for applying a force toward the support surface 324. In a manner described further below, individual plates of the segmented assembly can be joined together with the bonding material disposed therebetween.

Referring first to the base assembly 322, elongated support surface 324 is formed on an elongated beam member 330 herein secured to footing members 332. Upstanding support legs 340, herein secured to footing members 332, include ends 342 for supporting elongated reaction structure 326. Ends 342 allow reaction structure 326 to be removably coupled thereto. In the embodiment illustrated, elongated reaction structure 326 includes projecting flanges 346, which are received by ends 342. Apertures in the ends 342 and the flanges 346 receive coupling pins for forming a rigid connection. As appreciated-by those skilled in the art, the form of the elongated reaction structure 326 can be varied and the manner in which structure 326 is joined to base assembly 322 can be varied within the scope of the invention. For instance, other forms of couplings can be used such as clamps, bolts, etc.

In the embodiment illustrated, support surface 324 comprises a machined surface in order to ensure a flat or planar surface. Support surface 324 is secured to an elongated beam member such as a wide beam or “I” beam.

As illustrated in FIG. 9, inflatable member 328 can be elongated and in the embodiment illustrated comprises two hoses, such as fire hoses. A compressor 350 provides a compressible gas such as air to the inflatable members 328. Inflatable members 328 are disposed between plate or plates of the segmented assembly and reaction structure 326. When inflated the inflatable members 328 exert a downward force upon the plate or plates of the segmented assembly, and more importantly, a uniform pressure or force across the plate or plates and along the length thereof, which ensures uniform contact with the adhesive of the damping material. A suitable inflation pressure has been found to be 100 psi for one hour. However, the pressure and length of time the pressure is maintained can vary depending on the damping material and adhesive used.

Although illustrated wherein inflatable members 328 comprise elongated tubes, equivalent structures include a plurality of individual members placed adjacent each other along the length of the segmented assembly. Likewise, other forms of inflation fluids such as liquids can also be used.

Referring back to FIG. 5, segmented assembly 32 can be assembled as follows. Plates 32E and 32G are placed on support surface 324. Referring also to FIG. 10, the sides of machined surface 324 can provide one or more reference surfaces, or aligning blocks 351 can be provided to maintain a desired spacing of the plates 32E and 32G along the length of the support surface 324 to configure the position of edges 33. If desired, plates 32E and 32G can be temporarily secured to the base assembly 322 herein with clamps 352. Clamps 352 can be mounted in any suitable location on base assembly 322, herein on one or more of upstanding legs 340.

The adhesive damping material is then interposed between plates 32E, 32G and plate 32F. Inflatable members 328 and reaction structure are then placed above plate 32F. Inflatable members 328 are then inflated and pressure is maintained as required to obtain desired bonding between plates 32E, 32F and 32G. This subassembly is then removed and plates 32A, 32B and 32C are then bonded in a similar manner.

FIG. 10 illustrates connection of each of the subassemblies with plate 32D. In particular, the subassemblies are disposed on the sides of support surface 324 with portions of plates 32C and 32E resting thereon. Clamps 350 or other suitable fasteners can be used to maintain alignment. If desired additional alignment blocks 354 can also be used. The adhesive, damping material and plate 32D are then placed on plates 32C and 32E and suitable pressure is applied to complete the segmented assembly 32. Although described and illustrated above where plates 32F, 32B and 32D are placed on top of the other plates during assembly, plates 32F, 32B and 32D can be placed below the other plates during assembly. Likewise, the order of assembly can change, which may require different forms of base assembly 322 and/or reaction structure 326 in order to provide necessary clearance.

Although the present invention has been described with reference to preferred embodiments, workers skilled in the art will recognize that changes may be made in form and detail without departing from the spirit and scope of the invention.

What is claimed is:

1. A gantry system comprising:
a pair of rails;
supports for supporting the rails in a spaced-apart relationship; and
a bridge spanning between the rail and moveable on the rails, wherein the bridge comprises a perimeter wall formed of overlapping segments with damping material disposed therebetween.

2. The gantry system of claim 1 wherein the bridge further comprises a rigid plate and the perimeter wall comprises a U-shaped housing fastened to the rigid plate.

3. The gantry system of claim 2 and further comprising linear bearings that support a mast, the linear bearings being secured to the rigid plate.

4. The gantry system of claim 3 and further comprising a plurality of fasteners, each fastener passing through apertures of the rigid plate to secure the linear bearings and the U-shaped housing together on opposite sides of the rigid plate.

5. The gantry system of claim 3 and further comprising a plurality of flanges extending from the U-shaped housing for fastening the U-shaped housing to the rigid plate.

6. The gantry system of claim 5 wherein at least one of the flanges is adapted to fasten to a major surface of the rigid plate.

7. The gantry system of claim 6 and further comprising a plurality of fasteners, each fastener passing through apertures of the rigid plate to secure the linear bearings and the U-shaped housing together on opposite sides of the rigid plate.

8. The gantry system of claim 1 wherein the damping material comprises a viscoelastic damping material.

9. The gantry system of claim 8 wherein opposed major surfaces of the viscoelastic damping material are joined to each respective segment.

10. The gantry system of claim 1 wherein the bridge further comprises overtravel stops limiting relative movement of overlapping segments.

11. The gantry system of claim 10 wherein the overtravel stops comprises a rod secured to a segment secured to one of the overlapping segments and extending through a clearance aperture provided in said overlapping segment.

12. A gantry support structure comprising:
a damping structure comprising a plurality of overlapping segments forming at least a portion of a housing and having a plurality of fastening regions spaced apart about the housing with damping material disposed between the segments.

13. The gantry support structure of claim 12 and further comprising a rigid plate and wherein the housing is U-shaped.

14. The gantry support structure of claim 13 and further comprising a plurality of flanges extending from the housing for fastening the housing to the rigid plate.

15. The gantry support structure of claim 14 wherein at least one of the flanges is adapted to fasten to a major surface of the rigid plate.

16. The gantry support structure of claim 12 wherein the damping material comprises a viscoelastic damping material.

17. The gantry support structure of claim 16 wherein opposed major surfaces of the viscoelastic damping material are joined to each respective segment.

18. The gantry support structure of claim 13 and further comprising linear bearings that support a an element moveable relative to the support structure, the linear bearings being secured to the rigid plate.

19. The gantry support structure of claim 18 and further comprising a plurality of fasteners, each fastener passing through apertures of the rigid plate to secure the linear bearings and the U-shaped housing together on opposite sides of the rigid plate.

20. The gantry support structure of claim 13 wherein the housing further comprises overtravel stops limiting relative movement of overlapping segments.

21. The gantry support structure of claim 20 wherein the overtravel stops comprise a rod secured to a segment secured to one of the overlapping segments and extending through a clearance aperture provided in said overlapping segment.

22. A telescoping mast comprising:
a first longitudinal tube section and a moveable longitudinal tube section moveable relative to the first longitudinal tube section, wherein at least one of the first longitudinal tube section and the moveable longitudinal tube section include a perimeter wall formed of overlapping wall plates with a damping material disposed therebetween.

23. The telescoping mast of claim 22 wherein the damping material comprises a viscoelastic damping material.

24. The telescoping mast of claim 23 wherein opposed major surfaces of the viscoelastic damping material are adhesively joined to each respective wall plate.

25. The telescoping mast of claim 24 wherein each of the longitudinal tube sections comprise a rigid plate and a plurality of wall plates fastened together and to the rigid plate with a plurality of fasteners.

26. The telescoping mast of claim 22 and further comprising overtravel stops limiting relative movement of overlapping wall plates.

27. The telescoping mast of claim 26 wherein the overtravel stops comprise a rod secured to a plate secured to one of the overlapping plates and extending through a clearance aperture provided in said overlapping plate.

28. The telescoping mast of claim 22 wherein said at least one of the longitudinal tube section comprises first and second wall plates defining first and second spaced apart longitudinal edges, a rigid plate joined to the spaced apart longitudinal edges, a first L-shaped wall plate fastened to the first wall plate with damping material disposed therebetween, a third wall plate fastened with bolts to the first L-shaped wall plate and fastened to a second L-shaped wall plate with damping material disposed therebetween, a fourth wall plate fastened to the second L-shaped wall plate with bolts and fastened to the second wall plate with damping material disposed therebetween.

29. The telescoping mast of claim 22 wherein said at least one of the longitudinal tube section comprises first and second wall plates defining first and second spaced apart longitudinal edges, a rigid plate joined to the spaced apart longitudinal edges, a third wall plate fastened to first and second L-shaped wall plates with damping material disposed therebetween, a fourth wall plate fastened to the first wall plate and the first L-shaped wall plate with damping material disposed, and a fifth wall plate fastened to the second wall plate and the second L-shaped wall plate with damping material disposed therebetween.

30. The telescoping mast of claim 22 wherein said at one of the longitudinal tube section comprises a U-shaped housing having a plurality of sides and a plurality of corners, a rigid plate joined to the U-shaped housing, a plurality of regions placed around the U-shaped housing and spaced apart at a location proximate a middle of any of the plurality of side walls and proximate any of the plurality of corners.

31. A fixture for assembling segmented panels, the fixture comprising:
a base assembly having an elongated support surface;
an elongated reaction structure held fixed relative to the support surface; and
an elongated inflatable member disposed between the support surface and the reaction structure along the support surface for applying a force toward the support surface.

32. The fixture of claim 31 wherein the member comprises an elongated tube.

33. The fixture of claim 31 and further comprising a compressor coupleable to the tube to inflate the tube with a gas.

34. The fixture of claim 31 and further comprising a plurality of upstanding legs removably coupled to the elongated reaction structure to support the elongated reaction structure.

35. The fixture of claim 31 wherein the base assembly includes a reference surface on a side of the base assembly for defining a distance of assembly of the segmented panels.

36. The fixture of claim 35 wherein the base assembly includes a clamp to hold the segmented panel against the reference surface.

37. The fixture of claim 36 and further comprising a plurality of upstanding legs removably coupled to the elongated reaction structure to support the elongated reaction structure.

38. The fixture of claim 37 wherein a plurality of the legs each include a clamp to hold the segmented panel against the reference surface.

39. A method for assembling elongated segmented panels comprising:
placing an elongated panel assembly comprising two elongated panels and a bonding material between the elongated panels on an elongated support surface having a reaction surface opposite the elongated support surface;
disposing a inflatable member between the panel assembly and the reaction surface; and
pressurizing the inflatable member to obtain a uniform force along the elongated panel assembly.

40. The method of claim 39 wherein pressurizing the inflatable member includes inflating the inflatable member with a gas.

41. The method of claim 40 wherein the inflatable member comprises an elongated tube.

42. A gantry system comprising:
spaced apart guide rails;
a mast; and
a bridge extending between and supported on the guide rails, the bridge moveable along the guide rails, the bridge comprising a plate with a mounting surface for supporting the mast and a segmented assembly joined to the plate to form an enclosed perimeter with the plate without deforming the mounting surface when the segmented assembly is joined to the plate.

43. The gantry system of claim 42 wherein the segmented assembly is U-shaped with spaced apart sides having longitudinal edges joined to the plate, each of the edges being adjusted to correspond to a surface of the plate to which the segmented assembly is secured upon.

44. The gantry system of claim 43 wherein each of the sides include overlapping plates.

45. The gantry system of claim 44 wherein a damping material joins the overlapping plates together.

46. The gantry system of claim 43 wherein the longitudinal edges include flanges extending perpendicularly from each of the corresponding sides, the flanges secured to the plate.

47. The gantry system of claim 46 and further comprising guides joined to the mounting surface for guiding the mast along the plate, wherein fasteners securing the guides to the plate further secure the segmented assembly to the plate.

48. The gantry system of claim 42 and further comprising separate fasteners securing the segmented assembly to the plate.

49. A method for making a bridge for a gantry system, the method comprising:
obtaining a plate having a mounting surface for supporting a mast; and
securing a segmented assembly to the plate to form an enclosed perimeter with the plate without deforming the mounting surface when the segmented assembly is joined to the plate.

50. The method of claim 49 wherein securing includes using a plurality of separate fasteners.

51. The method of claim 49 and further comprising:
forming the segmented assembly to be U-shaped with spaced apart sides and longitudinal edges, each of the edges being adjusted to correspond to a surface of the plate to which the segmented assembly is secured upon.

52. The method of claim 51 wherein forming includes overlapping plates to form each of the sides.
